(19) United States
(12) Patent Application Publication
(10) Pub. No.: US 2019/0388381 A1
(43) Pub. Date: Dec. 26, 2019

(54) METHODS OF INHIBITING CISD PROTEIN-PLP COMPLEX FORMATION

(71) Applicants: Mary E. Konkle, Muncie, IN (US); Michael A. Menze, Louisville, KY (US); Werner J. Geldenhuys, Morgantown, WV (US)

(72) Inventors: Mary E. Konkle, Muncie, IN (US); Michael A. Menze, Louisville, KY (US); Werner J. Geldenhuys, Morgantown, WV (US)

(21) Appl. No.: 16/448,312

(22) Filed: Jun. 21, 2019

Related U.S. Application Data
(60) Provisional application No. 62/688,831, filed on Jun. 22, 2018.

(51) Int. Cl. A61K 31/341

(52) U.S. Cl. CPC A61K 31/341

(57) ABSTRACT

Methods of inhibiting formation of complexes of CDGSH iron-sulfur domain (CISD) protein and pyridoxal-5′-phosphate (PLP) are provided herein. More specifically, methods of inhibiting formation of CISD protein-PLP complexes include exposing a CISD protein to a compound with specific binding affinity for a designated lysine residue in the CISD protein. Inhibition of CISD protein-PLP complex formation is relevant to disease states associated with the CISD protein-PLP complex.

### CROSS REFERENCE TO RELATED APPLICATION

The present invention claims the benefit of priority to U.S. Provisional Patent Application Ser. No. 62/688,831 for ASSAY METHODS FOR CISD PROTEINS, filed Jun. 22, 2018, incorporated herein by reference.

### FIELD

Methods of inhibiting formation of complexes of CDGSH iron-sulfur domain (CISD) protein and pyridoxal-5′-phosphate (PLP) are provided herein. More specifically, methods of inhibiting formation of CISD protein-PLP complexes include exposing a CISD protein to a compound with specific binding affinity for a designated lysine residue in the CISD protein. Inhibition of CISD protein-PLP complex formation is relevant to disease states associated with the CISD protein-PLP complex.

### BACKGROUND

CISD proteins, such as mitoNEET and the nutrient autophagy factor 1 (NAF-1), are promising drug targets for several devastating diseases including type-2 diabetes, Parkinson's disease, and cancer. More specifically, the (dys)regulation of circulating free fatty acids linked to type-2 diabetes has been tied to mitoNEET through protein expression experiments in the ob/ob rodent model. MitoNEET has been implicated in the progression of Parkinson's Disease through the ubiquitination of mitoNEET by Parkin. Both mitoNEET and NAF-1 are implicated in the progression of breast cancer as determined by both cell culture and xenograft studies. Additionally, mutations in NAF-1 manifest as Wolfram Syndrome-2, which is characterized by early-childhood diabetes and a reduced life expectancy to only 30 years. Both knock-out and over-expression studies in animal models demonstrate that CISD proteins are key regulators of cellular energy, calcium, and iron homeostasis, making CISD proteins promising drug targets for diseases and conditions associated with misregulation thereof. However, the fundamental molecular mechanism for how CISD proteins induce or prevent metabolic dysfunctions is absent. This major knowledge gap hinders drug development and impedes treatments targeting CISD proteins. Biochemically, properties of a large number of CISD mutants have been characterized in vitro, but these techniques do not translate into assays to test the efficacy of possible drug molecules.

The discovery of CDGSH iron-sulfur domain (CISD) protein mitoNEET in 2004 was exciting not only because it represented a novel ligation pattern for [2Fe-2S] clusters in proteins, but also because it was identified from an effort to determine targets of the type-2 diabetes drug pioglitazone. MitoNEET was, therefore, quickly touted as a drug target. However, the lack of certain identification of the cellular function(s) of the CISD family members continues to foil drug development efforts. Structural characterization by X-ray crystallography of both, mitoNEET (cisd1) and NAF-1 (cisd2), showed that these two CDGSH family members are dimers and each monomer contains one [2Fe-2S] cluster ligated by three cysteine residues and one histidine residue. The two proteins have high sequence homology (66% identical) and a conserved fold (0.674 Å RMSD by backbone). Similar to Rieske proteins, which also contain [2Fe-2S] clusters but which are ligated by two cysteine and two histidine residues, mitoNEET and NAF-1 exhibit a pH-dependent reduction potential and are, therefore, postulated to operate through proton coupled electron transfer in a cellular environment. Furthermore, an observed instability of the cluster that is unique to CISD proteins has led to the hypothesis that these proteins not only serve as electron transport proteins but also as a donor of the cluster in a cytosolic iron-sulfur cluster assembly pathway.

The stability of the [2Fe-2S] cluster in mitoNEET is affected by pH, redox state of the iron, ligation of the iron, and the amino acids in the protein scaffold that are within five angstroms of the cluster. The cluster of mitoNEET, and to a lesser extent NAF-1, is unstable in an acidic environment in the oxidized state. Since mutating the ligating histidine to a cysteine residue confers pH-independent stability, the current model postulates that the protonation of the ligating histidine is responsible for the instability of the of the CISD proteins. This is confounding considering that Rieske proteins have two conserved ligating histidine residues, but yet have much greater cluster stability over a wide pH range. The supporting hypothesis is that a hydrogen-bonding network between the ligating histidine, a conserved solvent water molecule, and the εN of Lys55 from the other polypeptide chain of the dimer, that is unique to mitoNEET and NAF-1 proteins, is responsible for the instability of the cluster (FIGS. 1A and 1B). Further support for this hypothesis is provided by the mutagenesis result that cluster stability is conferred by the Lys55 to isoleucine mutation of mitoNEET.

The preponderance of structural information on CISD proteins is paired with a frustrating lack of functional clarity. For example, it has been well documented that knocking-out mitoNEET significantly lowers mitochondrial activity in heart, but increases respiration in adipocytes and hepatocytes, as measured by oxygen consumption. This result is confounding when one considers that the localization of mitoNEET is in the outer mitochondrial membrane with the cluster-binding domain facing into the cytosol as opposed to the location of the oxidative phosphorylation machinery (inner mitochondrial membrane). Furthermore, over-expression of mitoNEET in an obesity mouse model caused a significant accumulation of adipose tissue and lowered circulating lipids, but strikingly did not induce insulin insensitivity. Both NAF-1 and mitoNEET may play a role in the progression of breast cancer as indicated by knock-outs in a xenograft model, but how those proteins integrate into the cellular transformation process is unknown. Despite the high structural homology, evidence is mounting that mitoNEET and NAF-1 have distinct, in addition to overlapping, functions. NAF-1 is localized to the membrane of the endoplasmic reticulum and knock-out studies indicated a specific role of NAF-1 in regulating autophagy and lifespan. The mechanism is through the association of NAF-1 with Bcl-2 through Beclin-150. Some of the puzzling results, as well as the tissue-specific observations in the knock-out and overexpression studies, may be caused by differences in the PLP-mediated functional modifications on CISD proteins.

### SUMMARY

The instant disclosure identifies that the CISD proteins mitoNEET and NAF-1 bind pyridoxal-5′-phosphate (PLP) to a specific lysine residue in close proximity to the iron sulfur cluster. This newly found, and first, distinct binding site in mitoNEET and NAF-1 serves as a target for compounds to inhibit formation of CISD protein-PLP complexes to treat, inhibit, or palliate disease states arising from or dependent, at least in part, upon CISD protein-PLP complexes. Modulation of the reactivity of CISD proteins toward PLP with drug compounds will lead to novel treatment avenues for metabolic and neurological disorders. Furosemide, a compound with moderate affinity, will block the formation of the mitoNEET⋅PLP complex in a concentration dependent manner, detectable by monitoring the absorbance signal at λ=380 nm that is assigned to free PLP. As a negative control, pioglitazone, a compound that does not bind mitoNEET at Lys55, was found not to preclude the formation of mitoNEET⋅PLP complex.

In some embodiments, the present invention comprises a method for inhibiting a metabolic or neurological disease, including inhibiting formation of a CISD protein-PLP complex. In further embodiments, inhibiting comprises contacting the CISD protein with a compound that inhibits CISD protein-PLP complex formation in an amount effective to inhibit formation of the complex. In certain embodiments, the compound is furosemide. In some embodiments, the CISD protein is mitoNEET, NAF-1, or one of mitoNEET and NAF-1. In further embodiments, inhibiting comprises inhibiting intermolecular interactions between PLP and a lysine residue of mitoNEET. In certain embodiments, inhibiting comprises inhibiting intermolecular interactions between Lys55 of mitoNEET and PLP. In some embodiments, inhibiting comprises inhibiting intermolecular interactions between PLP and a lysine residue of NAF-1. In further embodiments, inhibiting comprises inhibiting intermolecular interactions between Lys78 of NAF-1 and PLP. In certain embodiments, inhibiting comprising inhibiting intermolecular interactions between PLP and a lysine residue of the CISD protein. In some embodiments, the metabolic or neurological disease state is one of diabetes, Parkinson's disease, cancer, Wolfram Syndrome-2, misregulation of cellular energy homeostasis, misregulation of cellular calcium homeostasis, and misregulation of cellular iron homeostasis. In further embodiments, the CISD protein includes an active site for binding PLP and wherein inhibiting comprises exposing the CISD protein to a compound that binds to the active site such that the compound blocks the active site, thus inhibiting formation of the CISD protein-PLP complex. In certain embodiments, the CISD protein is mitoNEET and the active site includes residue Lys55 of mitoNEET. In some embodiments, the CISD protein is NAF-1 and wherein the active site includes residue Lys78 of NAF-1.

In some embodiments, the present invention comprises a method for inhibiting formation of CISD protein-PLP complex including contacting a CISD protein with a compound that inhibits CISD protein-PLP complex formation in an amount effective to inhibit formation of the complex. In further embodiments, the CISD protein is mitoNEET and the compound has specific binding affinity for Lys55 residue of mitoNEET. In certain embodiments, the CISD protein is NAF-1 and the compound has specific binding affinity for Lys78 residue of NAF-1. In some embodiments, the compound is furosemide. In further embodiments, the CISD protein includes an active site for binding PLP and wherein said contacting comprises contacting the CISD protein with a compound that inhibits CISD protein-PLP complex by binding to the active site such that the compound blocks the active site from binding PLP.

In some embodiments, the present invention comprises a method for inhibiting formation of CISD protein-PLP complex comprising exposing a CISD protein with a molecule that inhibits intermolecular interactions between PLP and a lysine residue of the CISD protein in an amount effective to inhibit formation of the CISD protein-PLP complex.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

FIG. 1 displays a crystal structure of mitoNEET (2QH7) showing (A) the backbone of monomer A in dark grey and B in light grey and (B) a close-up view of the iron-sulfur cluster and the ligating residues of monomer A, and Lys55 of monomer B of mitoNEET. The coordinate covalent bonds and hydrogen bond network between Lys55, HOH532, and His87 are shown in dashed black lines.

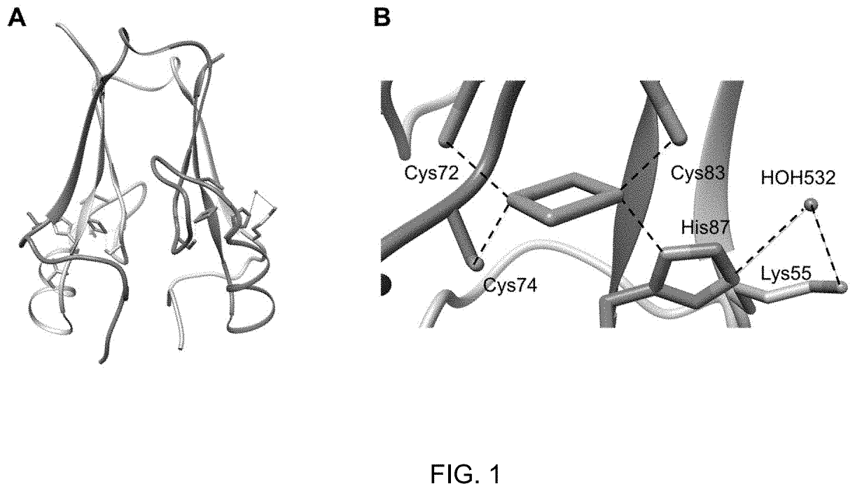

FIG. 2 displays absorbance spectra graphs depicting modification of mitoNEET (whose signal is subtracted from the data as a blank) by pyridoxal-5′-phosphate over time in the raw data (A) and difference spectra (B).

FIG. 3 displays absorbance spectra graphs showing (A) mitoNEET⋅PLP (red spectrum) reacted with 1 mM cysteine and (B) the degradation of the internal aldimine signal at 410 nm over 5000 sec.

FIG. 4 displays a photograph of an electrophoresis gel showing knock-down of mitoNEET in HepG2 cell clone 1. Both lanes were loaded with 40 μg of total protein.

FIG. 5 displays a chart showing overexpression of NAF-1 (gray bars) in HepG2 (open bars) cells results in a significant increase in respiration of permeabilized cells. “*” Indicates statistically significant differences between HepG2-CISD2 and HepG2 control (p<0.05).

FIG. 6 displays (A) a crystal structure of furosemide bound to mitoNEET, the non-covalent interactions shown in dashed black lines, and (B) a graph of absorbance over time displaying the kinetics of modification of mitoNEET by PLP with (square) or without (circle) furosemide shown by monitoring λ=435 nm.

FIG. 7 displays a graph showing mitoNEET denaturation curve (solid line) is shifted towards lower temperature in presence of ATP (dotted line) in 50 mM Tris-HCl, pH 7.5.

FIG. 8 displays a graph showing thermal shift assay results for mitoNEET with changing concentrations of adenosine monophosphate (AMP), adenosine diphosphate (ADP), and adenosine triphosphate (ATP).

FIG. 9 displays absorbance spectra graphs showing (A) the accumulation of signal at at λ=380 nm for unbound PLP as the concentration of furosemide increases indicating competitive binding and (B) the lack of change of the signal at λ=380 nm for unbound PLP as the concentration of AMP increases indicating no binding at Lys55 (negative control).

### DETAILED DESCRIPTION OF THE EXEMPLARY EMBODIMENTS

The embodiments disclosed below are not intended to be exhaustive or limit the disclosure to the precise forms disclosed in the following detailed description. Rather, the embodiments are chosen and described so that others skilled in the art may utilize their teachings.

CISD proteins readily react with PLP in a site-specific manner, but the physiologically-relevant substrate(s) and product(s) of the PLP-dependent reactions catalyzed by these proteins are presently unknown. In general, PLP-dependent enzymes catalyze a strikingly wide array of reactions on amino acid substrates including transamination, racemization, decarboxylation, and β-elimination. Each of these transformations requires three fundamental steps 1) formation of the internal aldimine with an active site lysine residue 2) formation of an external aldimine with the amino acid substrate 3) deprotonation of the external aldimine to form the carbanion intermediate that is stabilized by the electron sink cofactor. The subsequent mechanism depends on the reaction that is catalyzed. In the transamination mechanism, the collapse of the external aldimine by water produces the α-keto acid product and pyridoxamine. The second substrate, an α-keto acid, forms a second external aldimine that is hydrolyzed to the amino acid product and simultaneously reforms the internal aldimine with the active site lysine residue.

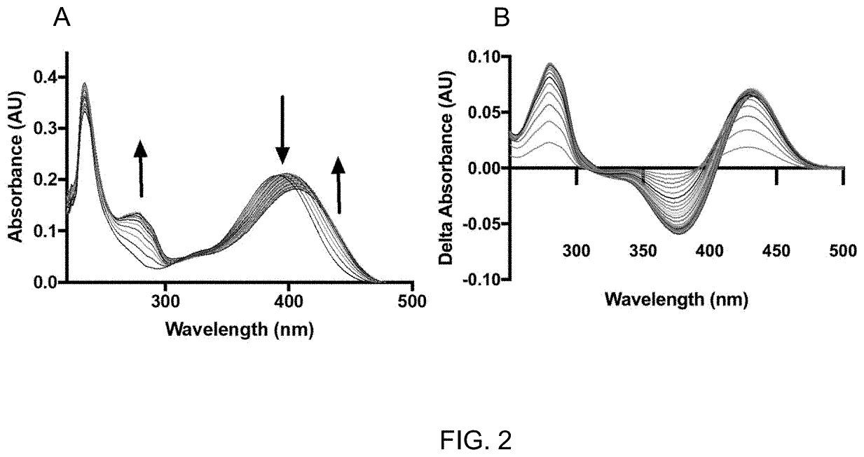

The initial discovery was made that CISD proteins bind PLP at a specific lysine residue that is in close proximity to the [2Fe-2S] cluster. PLP modifies the active site lysine residue of PLP-dependent enzymes forming the internal aldimine. In mitoNEET, PLP selectively modifies Lys55. While Lys55 was hypothesized to play a role in the reduction potential and stability of the [2Fe-2S] cluster in mitoNEET, its role as the binding site of PLP is novel to this work. MitoNEET was previously not known or classified as a PLP-dependent enzyme. Referring to FIG. 2, the formation of the mitoNEET⋅PLP and NAF-1⋅PLP complexes can be followed spectroscopically by the accumulation of a signal at λ=435 nm which is consistent with the ketoenamine tautomer and the attenuation of the signal at λ=380 nm which signifies the free PLP molecule. Concurrently with the increase of signal at λ=435 nm, an increase at λ=280 nm is also observed, but the chemical species contributing to this signal is currently unknown. In contrast to PLP, no reactivity of mitoNEET towards the non-phosphorylated pyridoxal (data not shown) was observed. We have found that Lys55 in mitoNEET is rapidly and selectively modified by PLP. Interestingly, the rate of reaction of NAF-1 protein being modified by PLP in PBS at pH 7.5 is even faster than the formation of PLP⋅mitoNEET under similar conditions (Table 1). An analogous lysine (Lys78) on NAF-1 is the analogous site for that protein. This indicates that CISD proteins in general exhibit PLP-dependent activities and further stresses the notion that the homology of fold observed in crystal structures among mitoNEET and NAF-1 is misleading about members' dynamic structure in solution and, therefore, specific function and/or kinetics and regulation.

TABLE 1
k_{obs} values for the modification of CISD proteins by PLP

| | k_{obs} values at | k_{obs} values at | k_{obs} values at |
|---|---|---|---|
| Protein | 280 nm | 435 nm | 380 nm |
| mitoNEET | 0.019 ± 0.001 | 0.019 ± 0.002 | 0.011 ± 0.003 |
| NAF-1 | 0.08 ± 0.01 | 0.08 ± 0.02 | 0.08 ± 0.02 |

Considering the presence of ten lysine residues in the soluble portion of mitoNEET, non-specific binding of PLP was a concern (PDB code 3EW0). Therefore, mitoNEET was treated with 2.0 molar equivalents of PLP over 20 minutes in phosphate buffered saline (PBS) at pH 7.5 and the reacted protein was analyzed by proteomics. In order to stabilize PLP modifications, the product(s) were treated with sodium borohydride to reduce the putative imine bond to the more stable amine. Next, the product(s) were degraded by tryptic digest, separated by liquid chromatography, analyzed by tandem mass spectrometry, and the data deconvoluted by proteomic analysis (courtesy of Indiana University Medical School Proteomics Core). As indicated in Table 2, Lys55 was clearly identified as the preferred site of modification by PLP in mitoNEET. Interestingly, Lys55 hydrogen bonds to the εN of the ligating residue His87 of the opposing protomer and has been implicated as a key residue in both cluster stability as well as reduction potential.

TABLE 2
Modification of mitoNEET lysine residues by pyridoxal-5′-phosphate
as identified as identified by proteomic analysis.

| Lysine Residue | | | |
|---|---|---|---|
| Position | Unmodified | Modified | Percent Modified |
| 55 | 27 | 25 | 48.1% |
| 68 | 646 | 53 | 7.6% |
| 79 | 62 | 4 | 6.1% |
| 89 | 78 | 2 | 2.5% |
| 104 | 200 | 3 | 1.5% |

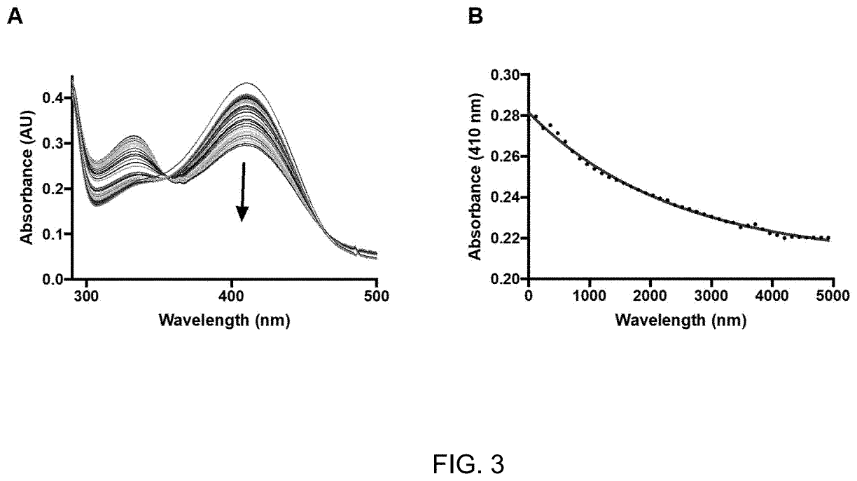

After PLP binding to the protein, the next step in many PLP-catalyzed enzymatic reactions is the formation of an external aldimine with one of several amino acid substrate such as cysteine, serine, alanine, or aspartate. Subsequently, an amazing diversity of chemistry can occur using this versatile coenzyme including transamination, racemization, and α,β-elimination. L-cysteine was selected as a first candidate substrate considering the previously shown reactivity of mitoNEET cysteine residues in the formation of mixed disulfide bonds. L-cysteine (0.5 mM) was added to the PLP⋅mitoNEET complex and the reaction was followed by spectrophotometry over 5000 sec. The degradation of the internal aldimine can be followed at λ=410 nm (FIG. 3) concurrent with the formation of a signal at λ=330 nm in a similar manner observed in SufS-SufE desulfuration enzyme complex. Interestingly, D-cysteine also reacts with the PLP⋅mitoNEET complex (albeit at a slower rate), whereas L-serine and L-phosphoserine (selected because of the propensity of mitoNEET to bind with negatively charged ligands) did not react (data not shown).

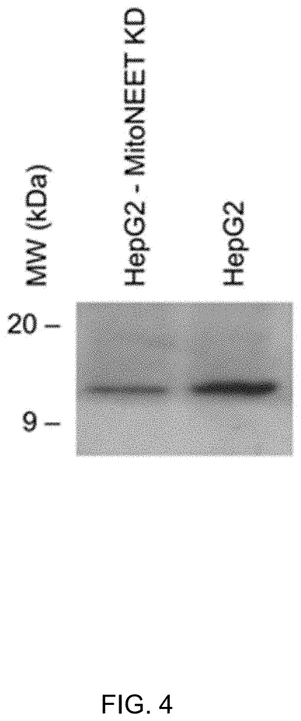

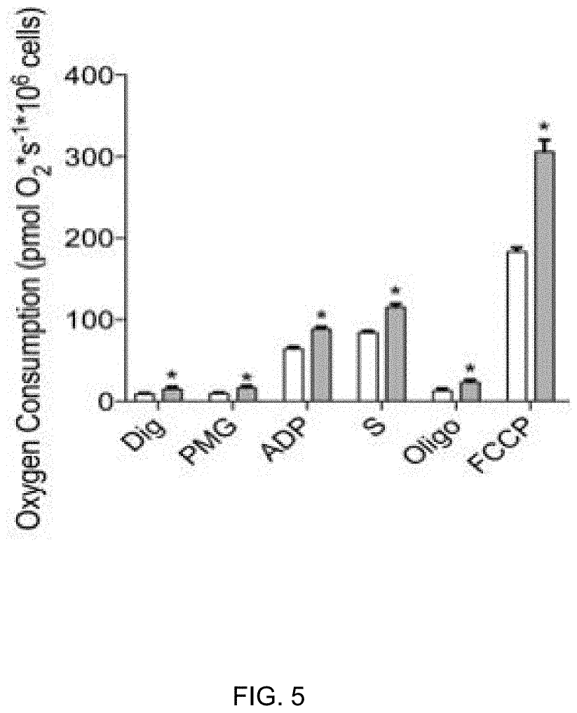

These interesting structure/function findings become much more impactful if they can be placed within the metabolic context of a cell. In order to understand the impact that CISD⋅PLP complexes have on cellular energetics and growth, we must first understand the energy profile in knock-down and overexpression of mitoNEET (FIG. 4) and NAF-1. Data show that overexpression of NAF-1 leads to increased levels of cellular respiration in presence of the FADH2-generating substrate (succinate, (S)), NADH-generating substrates (malate, glutamate, pyruvate (MGP)) and the increased respiration rate is maintained in presence of ADP. However, a dramatic increase of oxygen flux after addition of the chemical uncoupler FCCP is observed (control: 184.14 1 and CISD2: 306 pmol O₂*s*per million cells, FIG. 5).

Prior to this discovery of the CISD⋅PLP complex, three states of CISD proteins were known; holo oxidized (Fe³⁺— Fe³⁺), holo reduced (Fe³⁺— Fe²⁺), and the apo protein. Holo CISD⋅PLP introduces an additional state that needs to be evaluated within the context of the biological function of cluster donation/loss and/or reduction potential. It has been reported that the lability of the iron-sulfur cluster of CISD proteins is significantly higher as compared to the Rieske or ferredoxin proteins. Without being bound by theory, it is hypothesized that the presence of the ligating histidine is responsible and this assertion is supported by the result that mutagenesis of the ligating histidine to a cysteine increases cluster stability. Additionally, binding of the drug pioglitazone also stabilizes the metal cluster. PLP modification of the CISD proteins represents the first physiologically relevant event that may modulate cluster stability.

CISD proteins have a pH-dependent reduction potential, indicating proton-coupled electron transfer, that is likely tied to not only the discrete protonation state of the ligating histidine residue, but also perturbation of the hydrogen bonding networks that includes Lys55 of mitoNEET. It is known that the charges of the amino acids in both the near proximity, as well as in sites more removed from the metal cluster of the Rieske proteins, impacts the reduction potential in such proteins. Additionally, the chemical modification of a ligating histidine of Thermus thermophilus Rieske caused cluster reduction. In addition to the function(s) as a homodimer, CISD proteins have known protein-protein interactions both in vitro and in a cellular context. MitoNEET interacts with glutamate dehydrogenase 1 (GDH1), ferredoxin, (cytosolic) aconitase (IRP-1), and NAF-1. NAF-1, in contrast, is known to interact with Bcl-2 through Beclin150. Additionally, small molecules such as NADP(H), FMN, NL-1, and pioglitazone are known to bind to mitoNEET.

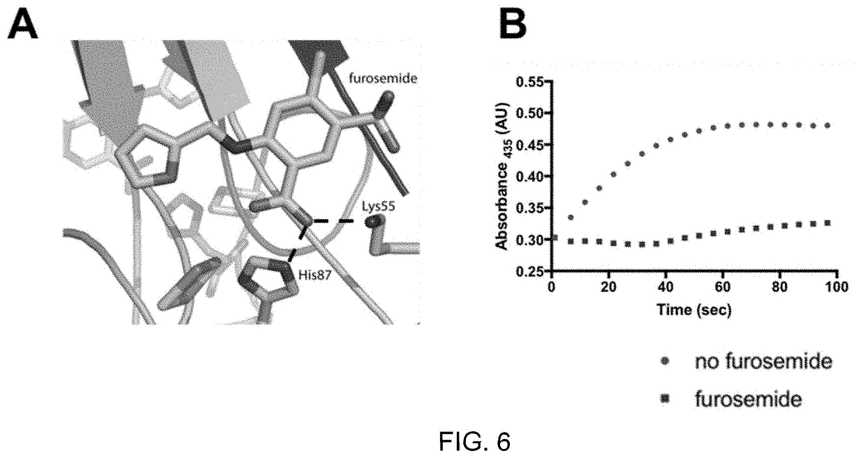

The identification of a preferred lysine, Lys55 in mitoNEET, for PLP binding is the first indication of an active site in CISD proteins. Other binding sites have been identified, but only from molecular modeling studies as there is no structure publicly available in the Protein Data Bank of a ligand-bound CISD protein at this time. However, a crystal structure accepted by the PDB (6DE9, entry on hold until publication, manuscript submitted) shows the medicinal compound furosemide bound to mitoNEET through either hydrogen-binding or ion-pairing with Lys55 (FIG. 6A). Since both PLP and furosemide appear to bind to mitoNEET through interaction(s) with Lys55, a competitive spectrophotometric assay was developed. The lack of formation of mitoNEET⋅PLP complex (signal at 2=435 nm) following pre-treatment with furosemide (FIG. 6B) confirms a discrete binding site for both small molecule inhibitors and the PLP coenzyme.

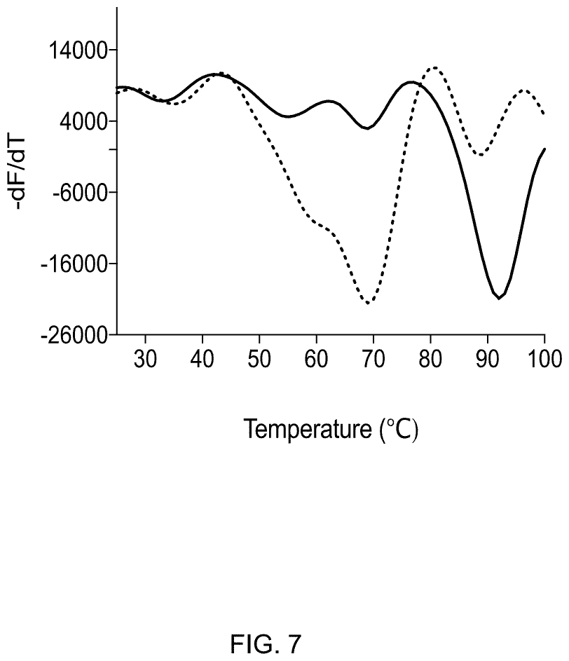

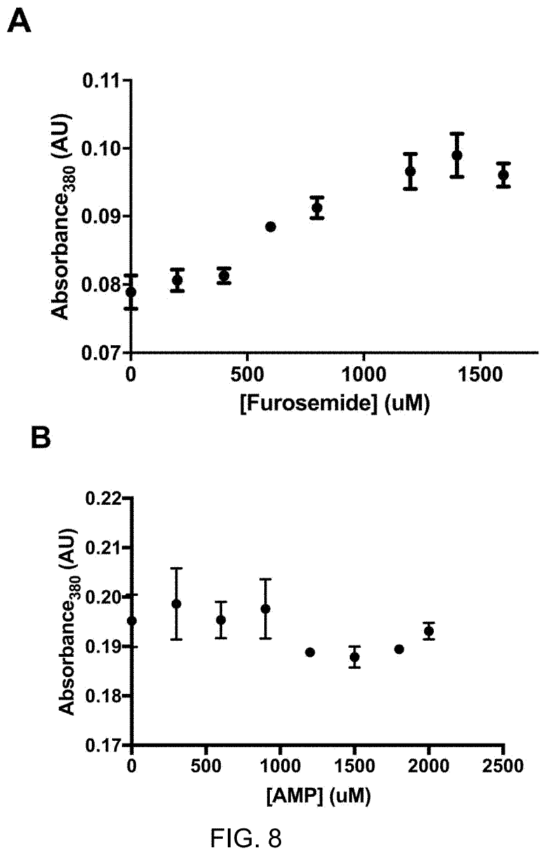

Thermal shift analysis reveals that physiological concentrations of ATP (8 mM) significantly destabilize mitoNEET. The assay is based on the principle that SYPRO Orange (SO), interacts with hydrophobic amino acids in a protein which become accessible to the dye upon thermally-induced unfolding of the polypeptide. Upon SO binding its fluorescence intensity increases which allows to measure the temperature at which CISD proteins unfold. By plotting the first derivative of the fluorescence emission as a function of temperature (−dF/dT), the melting temperature (Tm) is revealed, which is identified as the lowest point of this curve. We observed that mitoNEET is a highly stable protein at pH 7.5 with a Tm of 91.25° C. Surprisingly, in presence of ATP (neutralized to pH 7.5), the Tm of mitoNEET drops to 68° C. revealing a pronounced destabilizing effect (FIG. 7). This indicates some type of regulatory function of ATP for mitoNEET. Thermal shift analysis was also used to determine the effect of AMP, ADP, and ATP on mitoNEET thermal stability (FIG. 8). AMP and ADP do not significantly affect the thermal shift profile in a concentration dependent manner whereas ATP significantly destabilized mitoNEET.

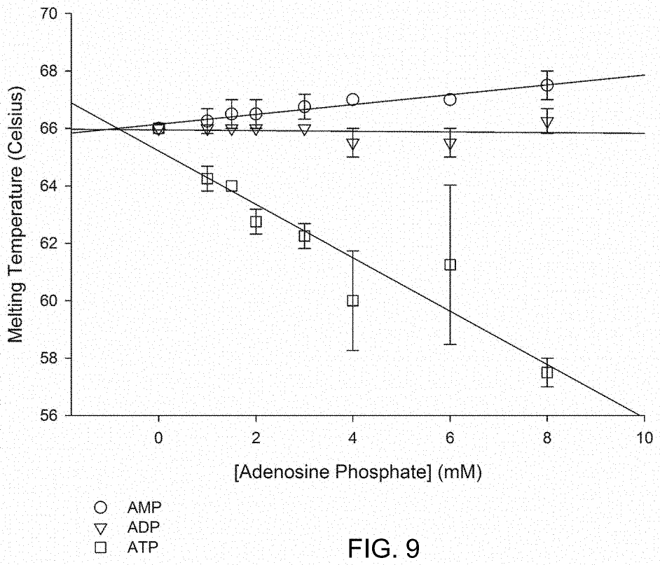

The binding of furosemide to mitoNEET relies on an interaction between the carboxyl group of furosemide and the terminal amino group of Lys55 (as seen in the crystal structure in FIG. 6A). The competitive screen was set up to monitor λ=380 nm (unbound PLP) as a function of increasing furosemide concentration (FIG. 9A). The increase in the signal at λ=380 nm indicates that the presence of furosemide prohibits the mitoNEET⋅PLP complex formation. The structural information indicates that the inhibition of formation is competitive, although it could be allosteric. In contrast, the presence of the negative control AMP does not correspond with an increase in the signal at λ=380 nm (FIG. 9B).

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

1) A method for inhibiting a metabolic or neurological disease, comprising:
inhibiting formation of a CISD protein-PLP complex.

2) The method of claim 1, wherein inhibiting comprises contacting the CISD protein with a compound that inhibits CISD protein-PLP complex formation in an amount effective to inhibit formation of the complex.

3) The method of claim 2, wherein the compound is furosemide.

4) The method of claim 1, wherein the CISD protein is mitoNEET.

5) The method of claim 4, wherein inhibiting comprises inhibiting intermolecular interactions between PLP and a lysine residue of mitoNEET.

6) The method of claim 5, wherein inhibiting comprises inhibiting intermolecular interactions between Lys55 of mitoNEET and PLP.

7) The method of claim 1, wherein the CISD protein is NAF-1.

8) The method of claim 7, wherein inhibiting comprises inhibiting intermolecular interactions between PLP and a lysine residue of NAF-1.

9) The method of claim 8, wherein inhibiting comprises inhibiting intermolecular interactions between Lys78 of NAF-1 and PLP.

10) The method of claim 1, wherein inhibiting comprising inhibiting intermolecular interactions between PLP and a lysine residue of the CISD protein.

11) The method of claim 1, wherein the metabolic or neurological disease state is one of diabetes, Parkinson's disease, cancer, Wolfram Syndrome-2, misregulation of cellular energy homeostasis, misregulation of cellular calcium homeostasis, and misregulation of cellular iron homeostasis.

12) The method of claim 1, wherein the CISD protein includes an active site for binding PLP and wherein inhibiting comprises exposing the CISD protein to a compound that binds to the active site such that the compound blocks the active site, thus inhibiting formation of the CISD protein-PLP complex.

13) The method of claim 12, wherein the CISD protein is mitoNEET and wherein the active site includes residue Lys55 of mitoNEET.

14) The method of claim 12, wherein the CISD protein is NAF-1 and wherein the active site includes residue Lys78 of NAF-1.

15) A method for inhibiting formation of CISD protein-PLP complex comprising contacting a CISD protein with a compound that inhibits CISD protein-PLP complex formation in an amount effective to inhibit formation of the complex.

16) The method of claim 15 wherein the CISD protein is mitoNEET and wherein the compound has specific binding affinity for Lys55 residue of mitoNEET.

17) The method of claim 15, wherein the CISD protein is NAF-1 and wherein the compound has specific binding affinity for Lys78 residue of NAF-1.

18) The method of claim 15, wherein the compound is furosemide.

19) The method of claim 15, wherein the CISD protein includes an active site for binding PLP and wherein said contacting comprises contacting the CISD protein with a compound that inhibits CISD protein-PLP complex by binding to the active site such that the compound blocks the active site from binding PLP.

20) A method for inhibiting formation of CISD protein-PLP complex comprising exposing a CISD protein with a molecule that inhibits intermolecular interactions between PLP and a lysine residue of the CISD protein in an amount effective to inhibit formation of the CISD protein-PLP complex.
